(19) United States
(12) Patent Application Publication
(10) Pub. No.: US 2024/0278070 A1
(43) Pub. Date: Aug. 22, 2024

(54) RESPIRATORY THERAPY DEVICES

(71) Applicant: SMITHS MEDICAL INTERNATIONAL LIMITED, Ashford (GB)

(72) Inventors: Ombeline Marone-Hitz, Ashford (GB); Jamie Daniel Perkins, Ashford (GB); Steven Mark Tupper, Hythe (GB); Stephen James Field, Canterbury (GB); Mohammad Qassim Mohammad Khasawneh, Canterbury (GB)

(73) Assignee: SMITHS MEDICAL INTERNATIONAL LIMITED, Ashford (GB)

(21) Appl. No.: 18/568,882

(22) PCT Filed: Jun. 29, 2022
(86) PCT No.: PCT/GB2022/000067

§ 371 Date: Dec. 11, 2023

(30) Foreign Application Priority Data
Jul. 6, 2021 (GB) 2109719.1

(51) Int. Cl. A63B 23/18; A61M 16/00; A61M 16/10; A61M 16/20

(52) U.S. Cl. CPC A63B 23/18; A61M 16/0006; A61M 16/1045; A61M 16/1055; A61M 16/106; A61M 16/208; A61M 2205/7509

(57) ABSTRACT

An oscillatory respiratory therapy device (100) has a rocker aim (105) that opens and closes a valve seat (106) by breathing through the device. The device (100) also includes a gas treatment arrangement including an HME (122) and filter (121) located such that both inhaled and exhaled gas passes through the HME and filter.

This invention relates to respiratory therapy devices of the kind having an inlet through which a user breathes, an opening to atmosphere, and a mechanism driven by breathing through the device to produce an oscillating resistance to breathing through the device.

Positive expiratory pressure (PEP) devices, that is, devices that present a resistance to expiration through the device, are now widely used to help treat patients suffering from a range of respiratory impairments, such as chronic obstructive pulmonary disease, bronchitis, cystic fibrosis, and atelectasis. More recently, devices that provide an alternating resistance to flow have been found to be particularly effective in that the expiration and vibration combine to stimulate upwards movement of secretions. One example of such devices is sold under the trade mark Acapella (a registered trade mark of Smiths Medical) by Smiths Medical and is described in U.S. Pat. Nos. 6,581,598, 6,776,159, 7,059,324 and 7,699,054. Other vibratory respiratory therapy devices are available, such as “Quake” manufactured by Thayer, “AeroPEP” manufactured by Monaghan, “TheraPEP” manufactured by Smiths Medical and “IPV Percussionater” manufactured by Percussionaire Corp. Alternative devices such as “CoughAssist” manufactured by Philips is also available. Respiratory therapy apparatus can instead provide an alternating resistance to flow during inhalation.

During use, the patient inhales and exhales by mouth through the therapy device, causing the natural warming and moistening carried out by the nose to be bypassed. As a result of this patients can suffer from dryness in their airways. Another perceived problem with such therapy devices is that the forceful exhalation through the device could cause particles from the airway to be driven out of the device and increase the risk of contamination of nearby people and surfaces by bacteria or viruses.

It is an object of the present invention to provide an alternative respiratory therapy device.

According to one aspect of the present invention there is provided a respiratory therapy device of the above-specified kind, characterised in that the device includes a gas treatment arrangement located in line with the mechanism such that at least air exhaled by the patient passes through the gas treatment arrangement before flowing to atmosphere.

The gas treatment arrangement is preferably located such that both exhaled and inhaled air passes through the gas treatment arrangement: The gas treatment arrangement may include a filter. The filter is preferably an electrostatic filter. The gas treatment device may additionally or alternatively include a heat and moisture exchange element. The mechanism to produce an alternating resistance to breathing preferably includes a displaceable member displaced by breathing through the device. The displaceable member preferably includes a rocker arm.

The gas treatment arrangement may be located between the mechanism and the opening to atmosphere such that exhaled air flows to the gas treatment arrangement after flowing through the mechanism.

Alternatively, the gas treatment arrangement may be located at the inlet of the device, such as in a removable mouthpiece.

According to another aspect of the present invention there is provided a mouthpiece for a device having the above alternative gas treatment arrangement.

A respiratory therapy device according to the present invention will now be described, by way of example, with reference to the accompanying drawings, in which:

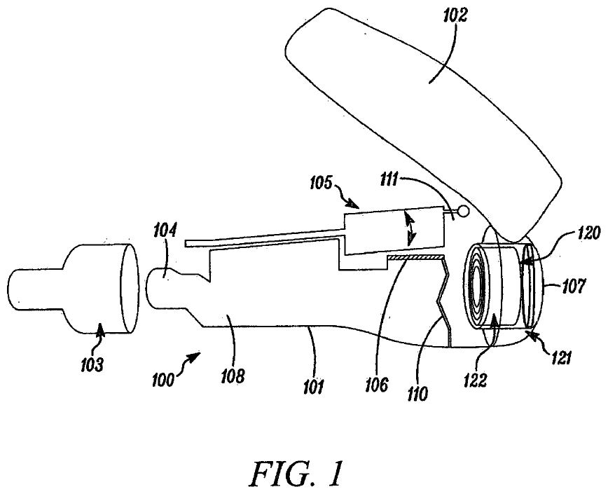

FIG. 1 is a simplified side elevation view of a first form of the device with its cover raised; and

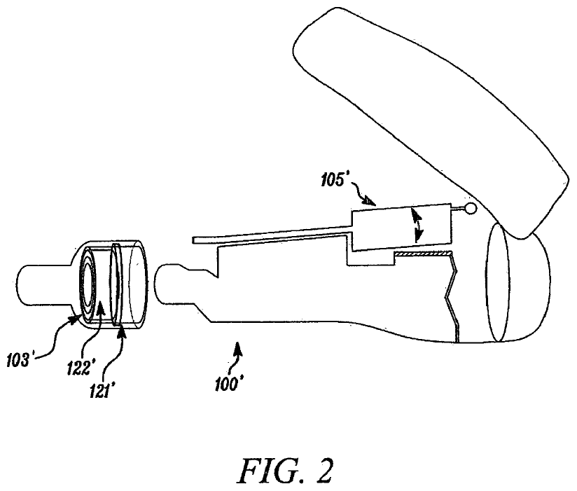

FIG. 2 is a simplified side elevation view of a second form of device with its cover raised.

With reference first to FIG. 1, the device 100 includes a housing 101 with a normally closed hinged cover 102 and a removable mouthpiece 103. The housing 101 has a patient inlet 104 at its left-hand end to which the mouthpiece 103 is fitted. The housing 101 contains a mechanism for producing an oscillating resistance to expiratory flow in the form of a magnetic rocker system 105 of the kind described in more detail in U.S. Pat. No. 6,581,598. A detailed understanding of the rocker system 105 is not needed to understand the present invention. The rocker system 105 normally closes a valve seat 106 opening from an air-flow passage 108 extending from the patient inlet 104 to an opening or outlet 107 at the opposite end of the housing 101: The air-flow passage 108 includes a one-way valve 110, such as a duck-bill valve, or flap valve, or some other conventional valve that allows flow on one direction. In particular, the valve 110 allows flow from the outlet 107 to the inlet 104 (during inhalation) but prevents or restricts flow in the opposite direction (during expiration). The valve 106 in the rocker system 105 opens from the air-flow passage 108 into a space 111 above the passage that is normally enclosed, when the cover 102 is in the lower, closed position during use. This space 111 communicates with the outlet 107.

It can be seen, therefore, that when the user inhales via the mouthpiece 103 and opening 104 air is drawn through the outlet 107 and the one-way valve 110 into the passage 108. The reduced pressure this creates in the passage 108 applies a pressure tending to draw the rocker system 105 further down against the valve seat 106, thereby further enhancing the normally closed state of this valve. When the user exhales through the mouthpiece 103 and inlet 104 this creates an increased pressure in the passage 108, which is prevented from flowing directly to the outlet 107 by the one-way valve 110. Instead, the pressure causes the valve 106 in the magnetic rocker system 105 to open, momentarily lifting the rocker arm and then allowing the pressure below the valve to drop. This allows the rocker arm to fall and close the valve seat 106 again until pressure builds up sufficiently to lift the rocker arm. In this way, the valve 106 alternately opens and closes, causing an oscillation or vibration in the air flow along the passage 108. This oscillation communicates with the user's respiratory system to produce a therapeutic vibration that helps loosen secretions.

As so far described the device 100 is substantially conventional. The device 100, however, is modified over conventional oscillatory therapy devices by the addition of a gas-treatment arrangement 120 disposed in the device in at least the expiratory flow path through the device. In the device shown the gas-treatment arrangement 120 is connected in both the inspiratory and expiratory flow paths between the outlet 107 and the one-way valve 110 (in the inspiratory path) and between the outlet and the rocker system valve 106 (in the expiratory flow path). The gas treatment arrangement 120 is located in the outlet 107 where its inner side is exposed to both air flowing out of the rocker valve 106 through the cavity 111 and where its inner side can allow air to flow through the gas-treatment arrangement 120 to the one-way valve 110. The gas treatment arrangement 120 could be either a filter or an HME element, or, as shown, the combination of both a filter 121 and an HME element 122. The filter 121 is preferably located on the external side of the HME 122 so that it filters exhaled air passing through the HME element and it filters inhaled air before it passes to the HME element. The filter 121 is preferably an electrostatic filter capable of trapping viruses and bacteria so that any contamination in breath exhaled by the user is filtered and the risk of spreading contamination or infection is reduced. Similarly, the filter 121 protects the user and the device 100 from external contamination when the user inhales. The HME element 122 may be of any conventional kind such as including a coil of corrugated paper treated with a hygroscopic salt, or a similarly treated foam disc. The HME element 122 receives all the air exhaled by the user so is warmed and moistened by passage of this air through the element. Similarly, all the air inhaled by the user through the device 100 must pass through the HME element 122 in the opposite direction so this is warmed and moistened by the heat and moisture stored in the element from the exhaled flow. In this way the drying effect on the user's during use of the device 100 is reduced.

In the device illustrated in FIG. 1 the filter 121 and HME element 122 are separate from one another and can be removed from the device 100 and replaced independently if the filter needs to be replaced at different intervals from the HME. Alternatively, the filter could be bonded with one or other of the faces of the HME to form a single unit that is removed and replaced together.

Instead of the HME and filter being mounted in the device at its outlet they could be mounted at the patient inlet, as shown in FIG. 2. A convenient way of doing this is to mount the HME 122′ and filter 121′ in the removable mouthpiece 103′. In the device shown in FIG. 2 the filter 121′ is located on the downstream side of the HME element 122′ during exhalation, that is, closer to the rocker mechanism 105′. This arrangement has the advantage of not requiring any change to the interior of the therapy device 100′ to accommodate the HME 122′ and filter 121′ since these are both incorporated in the mouthpiece 103′. It also makes it easier to replace the HME and filter. This arrangement, by reducing the moisture content of the exhaled breath flowing through the valve mechanism, could reduce contamination of the valve mechanism and enable the device to be used longer before it has to be replaced.

The two arrangements described above have a gas-treatment arrangement including both an HME and a filter. However, it would be possible to use just an HME element, where filtering was not required, or just a filter, where humidification was not required. Where the gas-treatment arrangement includes a filter but not an HME element it could be located in a gas-flow path that receives only expiratory gas flow, with inspiratory flow by-passing the filter. Other forms of gas treatment arrangement could be used.

1-11. (canceled)

12. A respiratory therapy device having an inlet through which a user breathes, an opening to atmosphere, and a mechanism driven by breathing through the device to produce an oscillating resistance to breathing through the device, characterised in that the device includes a gas treatment arrangement located in line with the mechanism such that at least air exhaled by the patient passes through the gas treatment arrangement before flowing to atmosphere.

13. The respiratory therapy device according to claim 12, characterised in that the gas treatment arrangement is located such that both exhaled and inhaled air passes through the gas treatment arrangement.

14. The respiratory therapy device according to claim 12, characterised in that the gas treatment arrangement includes a filter.

15. The respiratory therapy device according to claim 14, characterised in that the filter is an electrostatic filter.

16. The respiratory therapy device according to claim 12, characterised in that the gas treatment arrangement includes a heat and moisture exchange element.

17. The respiratory therapy device according to claim 12, characterised in that the mechanism to produce an alternating resistance to breathing includes a displaceable member displaced by breathing through the device.

18. The respiratory therapy device according to claim 17, characterised in that the displaceable member includes a rocker arm.

19. The respiratory therapy device according to claim 12, characterised in that the gas treatment arrangement is located between the mechanism and the opening to atmosphere such that exhaled air flows to the gas treatment arrangement after flowing through the mechanism.

20. The respiratory therapy device according to claim 12, characterised in that the gas treatment arrangement is located at the inlet of the device.

21. The respiratory therapy device according to claim 19, characterised in that the gas treatment arrangement is located in a removable mouthpiece.

22. A removable mouthpiece for a respiratory therapy device having an inlet through which a user breathes, wherein the device includes an opening to atmosphere, a mechanism driven by breathing through the device to produce an oscillating resistance to breathing through the device, and a gas treatment arrangement located in line with the mechanism such that at least air exhaled by the patient passes through the gas treatment arrangement before flowing to atmosphere.

24. The removable mouthpiece of claim 22, characterised in that the gas treatment arrangement is located between the mechanism and the opening to atmosphere such that exhaled air flows to the gas treatment arrangement after flowing through the mechanism.

25. The removable mouthpiece of claim 22, characterised in that the gas treatment arrangement is located in the removable mouthpiece.
